(19) United States
(12) Patent Application Publication
(10) Pub. No.: US 2026/0272360 A1
(43) Pub. Date: Sep. 17, 2026

(54) AUTOMATIC MEASUREMENT OF VENTRICULAR ACTIVATION TIME

(71) Applicant: AliveCor, Inc., Mountain View, CA (US)

(72) Inventors: David E. Albert, Oklahoma City, OK (US); Kim Norman Barnett, Mt. Tamborine (AU)

(21) Appl. No.: 19/563,969

(22) Filed: Mar. 11, 2026

Related U.S. Application Data
(60) Provisional application No. 63/770,946, filed on Mar. 12, 2025.

(51) Int. Cl. A61B 5/349; A61B 5/00; A61B 5/252; A61B 5/254; A61B 5/257; A61B 5/274; A61B 5/282; A61B 5/332; A61B 5/339

(52) U.S. Cl. CPC A61B 5/349; A61B 5/252; A61B 5/254; A61B 5/257; A61B 5/274; A61B 5/282; A61B 5/332; A61B 5/339; A61B 5/6823; A61B 5/6824; A61B 5/6828; A61B 5/6832; A61B 5/7475; A61B 2562/166; A61B 2562/222; A61B 2562/227

(57) ABSTRACT

Embodiments of the present disclosure provide a reduced lead 12-lead ECG device having a set of electrodes disposed along a cable to measure electrical activity of a user's heart at e.g., the RA, LA, LL, V1 and V6 positions. The cable is shaped in such a manner that an electrode of the set of electrodes can measure electrical activity of the user's heart at the V6 position without a lead extender, and thus can obtain the leads V1 and V6 simultaneously to generate a V6-V1 interpeak interval. The V6-V1 interpeak interval can help differentiate between different types of ventricular conduction system capture, evaluate a level of ventricular conduction system capture, and/or identify transitions between capture types.

### CROSS REFERENCE TO RELATED APPLICATIONS

This application claims priority to U.S. Provisional Application No. 63/770,946, filed on Mar. 12, 2025, entitled “AUTOMATIC MEASUREMENT OF VENTRICULAR ACTIVATION TIME,” the contents of which is incorporated herein by reference in its entirety.

### TECHNICAL FIELD

The present disclosure relates to medical devices, systems, and methods and in particular, to a small form-factor device for providing electrocardiogram (ECG) monitoring.

### BACKGROUND

Cardiovascular diseases are the leading cause of death in the world. In 2008, 30% of all global death can be attributed to cardiovascular diseases. It is also estimated that by 2030, over 23 million people will die from cardiovascular diseases annually. Cardiovascular diseases are prevalent across populations of first and third world countries alike, and affect people regardless of socioeconomic status.

Arrhythmia is a cardiac condition in which the electrical activity of the heart is irregular or is faster (tachycardia) or slower (bradycardia) than normal. Although many arrhythmias are not life-threatening, some can cause cardiac arrest and even sudden cardiac death. Indeed, cardiac arrhythmias are one of the most common causes of death when travelling to a hospital. Atrial fibrillation (A-fib) is the most common cardiac arrhythmia. In A-fib, electrical conduction through the ventricles of heart is irregular and disorganized. While A-fib may cause no symptoms, it is often associated with palpitations, shortness of breath, fainting, chest pain or congestive heart failure and also increases the risk of stroke. A-fib is usually diagnosed by taking an electrocardiogram (ECG) of a subject. To treat A-fib, a patient may take medications to slow heart rate or modify the rhythm of the heart. Patients may also take anticoagulants to prevent stroke or may even undergo surgical intervention including cardiac ablation to treat A-fib. In another example, an ECG may provide decision support for Acute Coronary Syndromes (ACS) by interpreting various rhythm and morphology conditions, including Myocardial Infarction (MI) and Ischemia.

Often, a patient with A-fib (or other type of arrhythmia) is monitored for extended periods of time to manage the disease. For example, a patient may be provided with a Holter monitor or other ambulatory electrocardiography device to continuously monitor the electrical activity of the cardiovascular system for e.g., at least 24 hours. Such monitoring can be critical in detecting conditions such as acute coronary syndrome (ACS), among others.

The American Heart Association and the European Society of Cardiology recommends that a 12-lead ECG should be acquired as early as possible for patients with possible ACS when symptoms present. Prehospital ECG has been found to significantly reduce time-to-treatment and shows better survival rates. The time-to-first-ECG is so vital that it is a quality and performance metric monitored by several regulatory bodies. According to the national health statistics for 2015, over 7 million people visited the emergency department (ED) in the United States (U.S.) with the primary complaint of chest pain or related symptoms of ACS. In the US, ED visits are increasing at a rate of or 3.2% annually and outside the U.S. ED visits are increasing at 3% to 7%, annually.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the invention are utilized, and the accompanying drawings of which:

FIG. 1 is a diagram illustrating electrocardiogram (ECG) waveforms, in accordance with some embodiments of the present disclosure.

FIG. 2 illustrates a single dipole heart model with a 12-lead set represented on a hex axial system, in accordance with some embodiments of the present disclosure.

FIG. 3A is a diagram illustrating an ECG monitoring device, in accordance with some embodiments of the present disclosure.

FIG. 3B is a diagram illustrating a computing device of the ECG monitoring device of FIG. 3A, in accordance with some embodiments of the present disclosure.

FIG. 3C illustrates the ECG monitoring device of FIG. 3A in operation, in accordance with some embodiments of the present disclosure.

FIG. 3D. illustrates a hardware diagram of the ECG monitoring device of FIG. 3A, in accordance with some embodiments of the present disclosure.

FIG. 3E. illustrates the ECG monitoring device of FIG. 3A in operation, in accordance with some embodiments of the present disclosure.

FIG. 3F illustrates an example graphical user interface (GUI) for displaying and analyzing a V6-V1 interpeak interval, in accordance with some embodiments of the present disclosure.

FIG. 3G illustrates three types of capture during cardiac pacing observed in a patient during pacemaker implantation, in accordance with some embodiments of the present disclosure.

FIG. 4A is a diagram illustrating an ECG monitoring device, in accordance with some embodiments of the present disclosure.

FIG. 4B is a diagram illustrating an ECG monitoring device, in accordance with some embodiments of the present disclosure.

FIG. 4C illustrates a hardware diagram of the ECG monitoring device of FIG. 4A, in accordance with some embodiments of the present disclosure.

FIG. 4D illustrates a hardware diagram of the ECG monitoring device of FIG. 4B, in accordance with some embodiments of the present disclosure.

FIG. 4E illustrates the ECG monitoring device of FIG. 4A in operation, in accordance with some embodiments of the present disclosure.

FIG. 5 is a block diagram of an example computing device that may perform one or more of the operations described herein, in accordance with some embodiments of the present disclosure.

### DETAILED DESCRIPTION

It is to be understood that the present disclosure is not limited in its application to the details of construction, experiments, exemplary data, and/or the arrangement of the components set forth in the following description. The embodiments of the present disclosure are capable of other embodiments or of being practiced or carried out in various ways. Also, it is to be understood that the terminology employed herein is for purpose of description and should not be regarded as limiting.

In the following detailed description of embodiments of the present disclosure, numerous specific details are set forth in order to provide a more thorough understanding of the disclosure. However, it will be apparent to one of ordinary skill in the art that the concepts within the disclosure can be practiced without these specific details. In other instances, well-known features have not been described in detail to avoid unnecessarily complicating the description.

An electrocardiogram (ECG) provides a number of ECG waveforms that represent the electrical activity of a person's heart. An ECG monitoring device may comprise a set of electrodes for recording these ECG waveforms (also referred to herein as “taking an ECG”) of the patient's heart. The set of electrodes may be placed on the skin of the patient in multiple locations and the electrical signal (ECG waveform) recorded between each electrode pair in the set of electrodes may be referred to as a lead. Varying numbers of leads can be used to take an ECG, and different numbers and combinations of electrodes can be used to form the various leads. Example numbers of leads used for taking ECGs are 1, 2, 6, and 12 leads.

The ECG waveforms (each one corresponding to a lead of the ECG) recorded by the ECG monitoring device may comprise data corresponding to the electrical activity of the person's heart. A typical heartbeat may include several variations of electrical potential, which may be classified into waves and complexes, including a P wave, a QRS complex, a T wave, and a U wave among others, as is known in the art. Stated differently, each ECG waveform may include a P wave, a QRS complex, a T wave, and a U wave among others, as is known in the art. The shape and duration of these waves may be related to various characteristics of the person's heart such as the size of the person's atrium (e.g., indicating atrial enlargement) and can be a first source of heartbeat characteristics unique to a person. The ECG waveforms may be analyzed (typically after standard filtering and “cleaning” of the signals) for various indicators that are useful in detecting cardiac events or status, such as cardiac arrhythmia detection and characterization. Such indicators may include ECG waveform amplitude and morphology (e.g., QRS complex amplitude and morphology), R wave-ST segment and T wave amplitude analysis, and heart rate variability (HRV), for example.

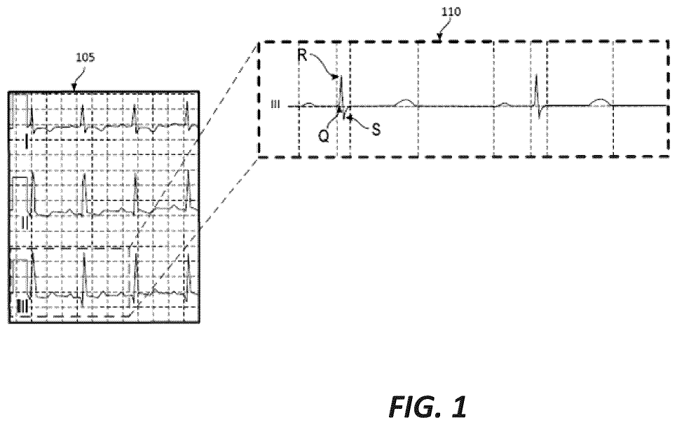

As noted above, ECG waveforms are generated from measuring multiple leads (each lead formed by a different electrode pair), and the ECG waveform obtained from each different electrode pair/lead may be different/unique (e.g., may have different morphologies/amplitudes). This is because although the various leads may analyze the same electrical events, each one may do so from a different angle. FIG. 1A illustrates a view 105 of an ECG waveform detected by each of 3 leads (I, II, and III) when a 3-lead ECG is taken as well as an exploded view 110 of the ECG waveform measured by lead III illustrating the QRS complex. As shown, the amplitudes and morphologies of the ECG waveform taken from leads I-III are all different, with the ECG waveform measured by lead III having the largest amplitude and the ECG waveform measured by lead I having the smallest amplitude.

There are different “standard” configurations for electrode placement that can be used to place electrodes on the patient. For example, an electrode placed on the right arm can be referred to as RA. The electrode placed on the left arm can be referred to as LA. The RA and LA electrodes may be placed at the same location on the left and right arms, preferably near the wrist in some embodiments. The leg electrodes can be referred to as RL for the right leg and LL for the left leg. The RL and LL electrodes may be placed on the same location for the left and right legs, preferably near the ankle in some embodiments. Lead I is typically the voltage between the left arm (LA) and right arm (RA), e.g. I=LA−RA. Lead II is typically the voltage between the left leg (LL) and right arm (RA), e.g. II=LL−RA. Lead III is the typically voltage between the left leg (LL) and left arm (LA), e.g. III=LL−LA. Augmented limb leads can also be determined from RA, RL, LL, and LA. The augmented vector right (aVR) lead is equal to RA−(LA+LL)/2 or −(I+II)/2. The augmented vector left (aVL) lead is equal to LA−(RA+LL)/2 or I-II/2. The augmented vector foot (aVF) lead is equal to LL−(RA+LA)/2 or II−I/2.

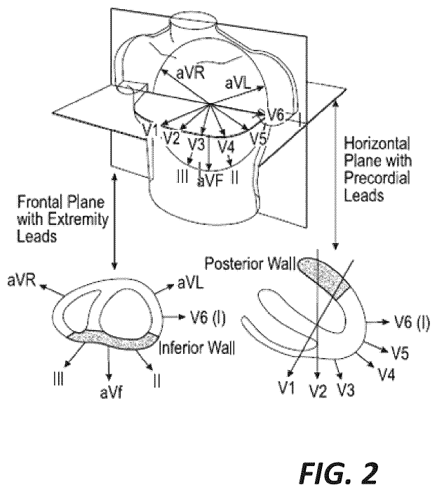

FIG. 2 illustrates a single dipole heart model 115 with a 12-lead set comprising the I, II, III, aVR, aVL, aVF, V1, V2, V3, V4, V5, and V6 leads, all represented on a hexaxial system. The heart model 115 assumes a homogeneous cardiac field in all directions that only changes magnitude and direction with the cycle time. As illustrated in FIG. 2, there are 2 orthogonal planes, the frontal plane and the horizontal plane. Inside each plane, there are several leads to cover the whole plane. In the frontal plane, there are 2 independent leads I and II, and 4 other derived leads III, aVR, aVL, and aVF, each 30 degrees apart. The reason the frontal plane has 2 independent leads is that they are far-field leads, each of which can cover a wider perspective but provide less detail, like a wide-angle camera lens. In the horizontal plane, there are normally 6 independent leads which are all closer to the heart than limb leads and may be referred to as near-field leads. Following the same analogy of a camera, the near-field leads may behave like a zoom-lens that covers less perspective, but with more accuracy towards local activity like ischemia and infarction. The two orthogonal planes are related by using a synthetic reference point formed by Leads I & II, called the Wilson-Central-Terminal (WCT). It is defined as RA+LA+RL/3 but given that both Lead I and II are recorded with reference to the RA so that the voltage of the RA can be considered zero, the WCT (VW) can be calculated using the RA as the reference for both Leads I & II (thus, assuming it to have zero potential) as: Lead I+Lead II/3.

It should be noted that a set of two or more leads may be transformed to generate a full, 12-lead ECG. Such transformation may be performed using a machine learning model (e.g., a neural network, deep-learning techniques, etc.). The machine learning model may be trained using 12-lead ECG data corresponding to a population of individuals. The data, before being input into the machine learning model, may be pre-processed to filter the data in a manner suitable for the application. For example, data may be categorized according to height, gender, weight, nationality, etc. before being used to train one or more machine learning models, such that the resulting one or models are finely-tuned the specific types of individuals. In a further embodiment, the machine learning model may be further trained based on a user's own ECG data, to fine-tune and personalize the model even further to decrease any residual synthesis error.

Cardiac pacing is a medical procedure that uses electrical impulses to regulate the heartbeat. In cardiac pacing, leads are implanted directly into the heart's intrinsic electrical pathways (e.g., the His bundle or left bundle branch area) to restore natural physiological activation. Conduction system pacing (CSP) is a type of cardiac pacing that targets the electrical conduction system of the heart. It aims to improve the rhythm and coordination of the heart by stimulating the ventricular conduction system directly, rather than the heart muscle itself. CSP, such as His bundle pacing (HBP) and left bundle branch pacing (LBBP), has the advantage of avoiding the electrical (and thereby mechanical) desynchronization induced by myocardial pacing by utilizing the intrinsic conduction system to activate the ventricles. The extent to which ventricular conduction system tissue is stimulated may be referred to as capture of the ventricular conduction system tissue. Analysis of an electrocardiogram (ECG) is crucial for confirming capture of ventricular conduction system tissue, which is a prerequisite for successful CSP at implantation and follow-up. However, evaluating the level of capture of the ventricular conduction system tissue can be challenging due to non-selective (ns) capture, which refers to the simultaneous activation/stimulation of the ventricular conduction system and the adjacent myocardium. This problem is exacerbated in LBB pacing where specific electrocardiogram (ECG) criteria are lacking.

One solution to this problem involves the use of the V6-V1 interpeak interval, which can be read from an ECG and is a helpful criterion for differentiating between different types of ventricular conduction system capture (e.g., between left bundle branch (LBB) capture and left ventricular septal myocardium (LVS) capture), evaluating a level of ventricular conduction system capture, and/or identifying transitions between capture types. The V6-V1 interpeak interval is the time between the R wave peak in the V6 lead and the terminal R wave peak in the V1 lead.

Many current ambulatory ECG devices, such as Holter monitors, can measure the V6-V1 interpeak interval. However, these are typically bulky and difficult to implement in an expedient manner. Although smaller form factor ECG measurement devices are known, many of these are intended to measure only one V lead or no V leads (e.g., due to size and other constraints). In addition, many such devices may have difficulty reaching the V6 position and/or are unable to measure the V1 and V6 leads simultaneously.

Embodiments of the present disclosure address the above and other issues by providing a reduced lead 12-lead ECG device having a set of electrodes disposed along a cable to measure electrical activity of a user's heart at e.g., the RA, LA, LL, V1 and V6 positions. The cable is shaped in such a manner that an electrode of the set of electrodes can measure electrical activity of the user's heart at the V6 position without a lead extender, and thus can obtain the leads V1 and V6 simultaneously to generate the V6-V1 interpeak interval. The V6-V1 interpeak interval can help differentiate between different types of ventricular conduction system capture, evaluate a level of ventricular conduction system capture, and/or identify transitions between capture types. The reduced lead 12-lead ECG device is also smaller and less cumbersome than a traditional 12-lead ECG device and the reduced lead set creates less radiographic obstruction during the electrode placement. Embodiments of the present disclosure are described with respect to measuring the V6-V1 interpeak interval for example purposes but are not limited in this way, and may be used to measure other parameters/criteria that can help differentiate between different types of ventricular conduction system capture, evaluate a level of ventricular conduction system capture, and/or identify transitions between capture types.

In some embodiments, the reduced lead 12-lead ECG device may have a set of electrodes disposed along the cable to measure electrical activity of a user's heart at e.g., the RA, LA, LL, V1 and V4 positions. The reduced lead 12-lead ECG device may utilize a lead extender to switch the position of an existing electrode (e.g., the V4 electrode) of the set of electrodes so that the existing electrode can measure electrical activity of the user's heart at the V6 position. In some embodiments, the lead extender instead allows for connection of an additional electrode (i.e., an electrode in addition to the set of electrodes) that can measure electrical activity of the user's heart at the V6 position to the cable. In this way, the reduced lead 12-lead ECG device can obtain leads V1 and V6 simultaneously to generate a V6-V1 interpeak interval, which can help differentiate between different types of ventricular conduction system capture, evaluate a level of ventricular conduction system capture, and/or identify transitions between capture types. The reduced lead 12-lead ECG device is also smaller and less cumbersome than a traditional 12-lead ECG device and the reduced lead set creates less radiographic obstruction during the electrode placement.

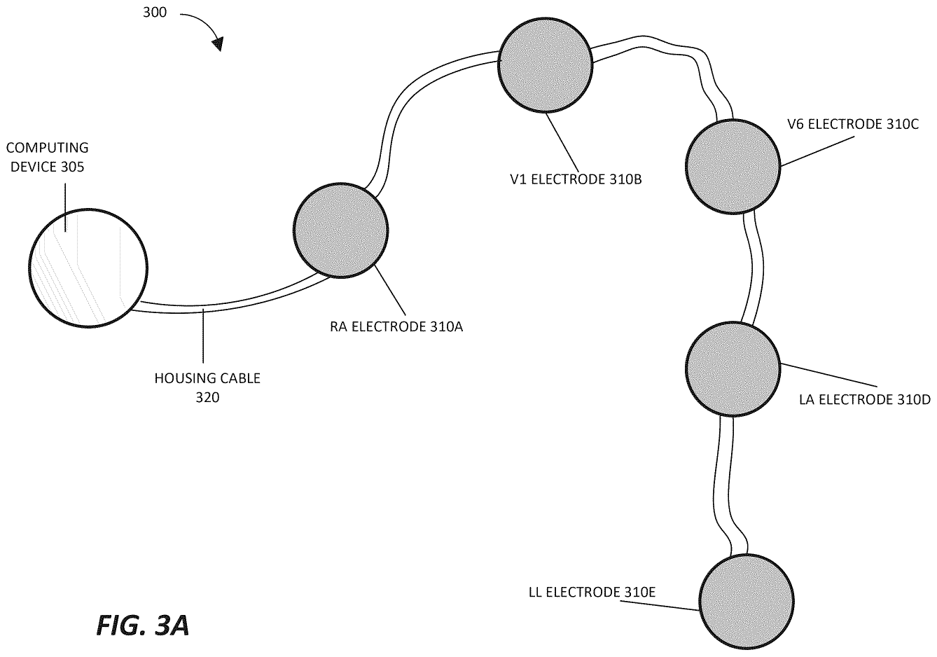

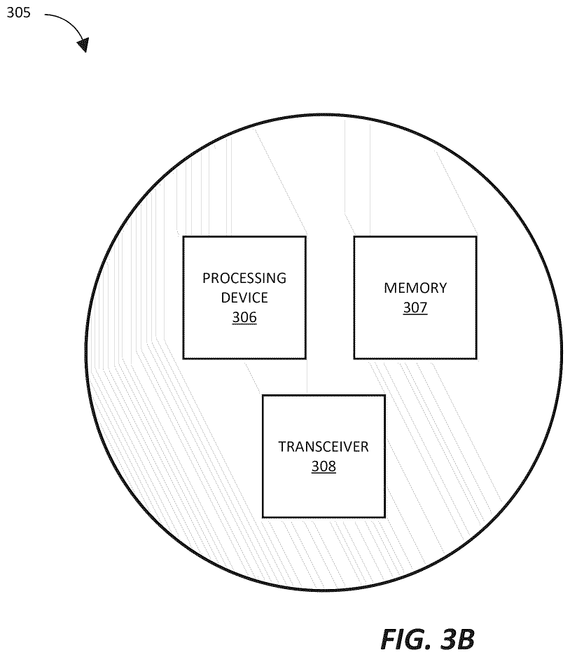

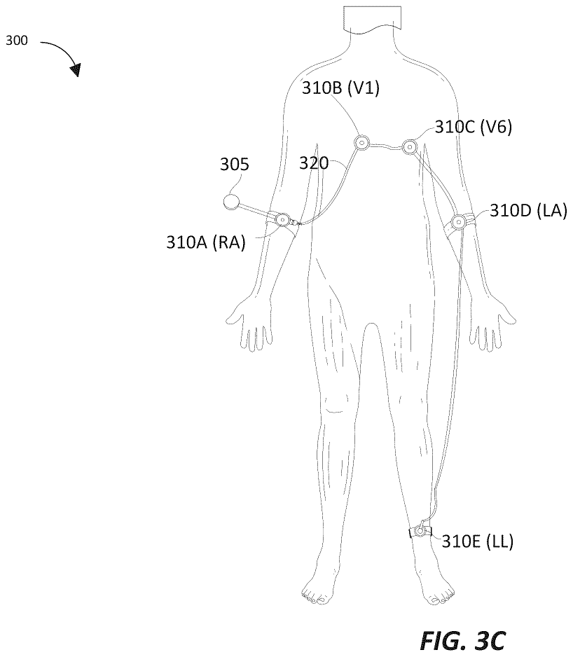

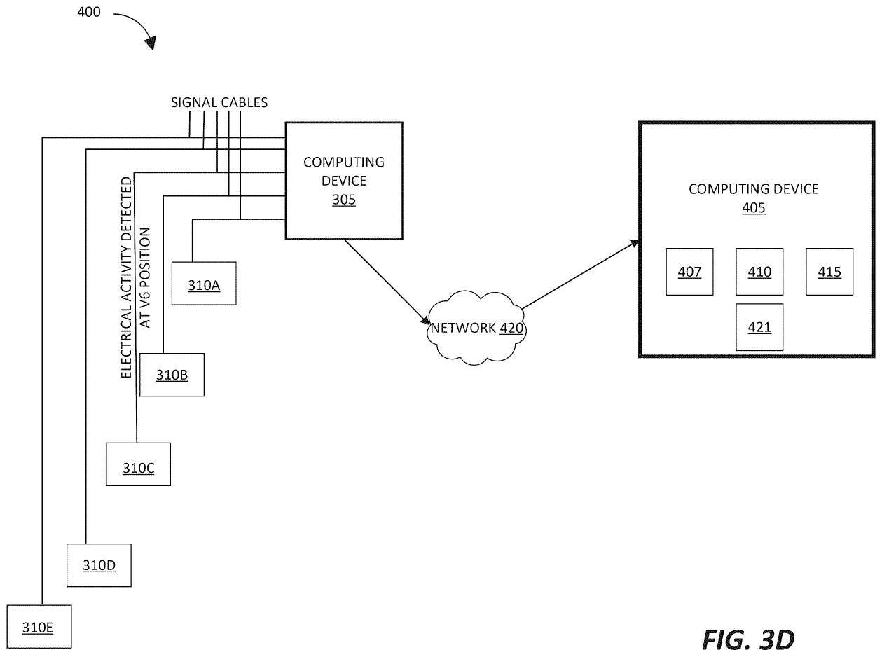

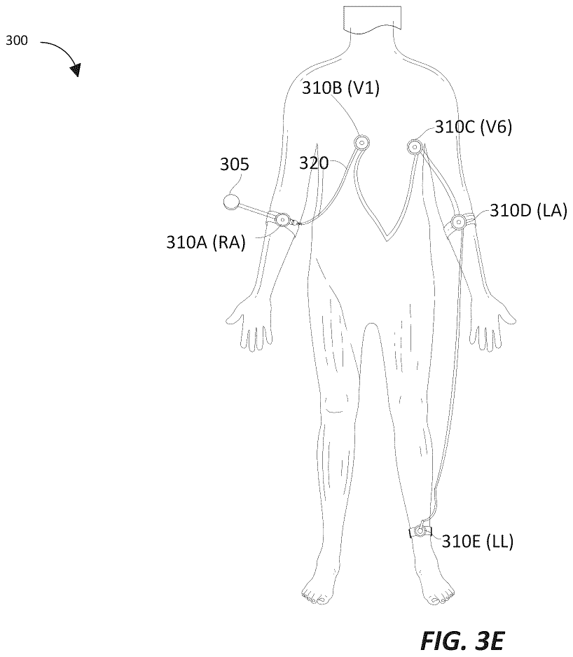

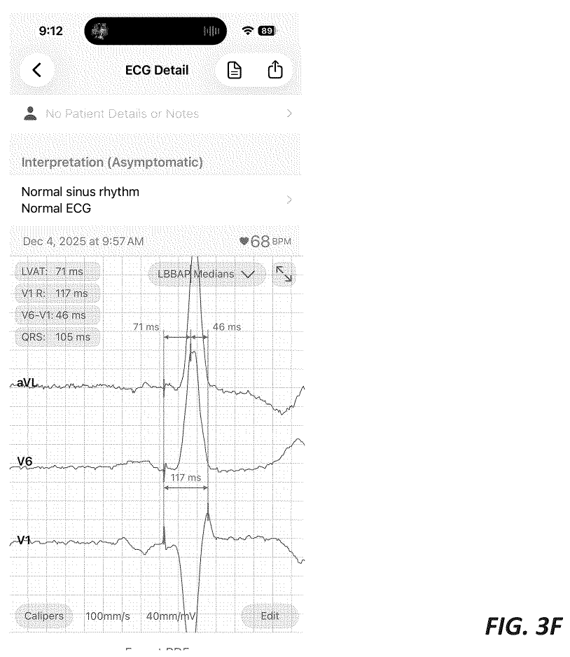

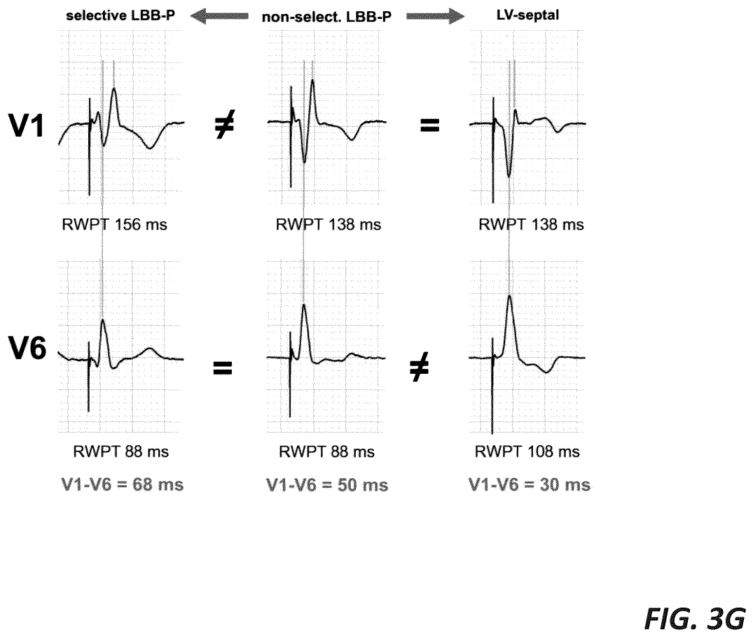

FIG. 3A shows an ECG monitoring device 300 in accordance with some embodiments of the present disclosure. The ECG monitoring device 300 may comprise a computing device 305, a housing cable 320, and a set of electrodes 310A-310E. Each of the electrodes 310 as well as the computing device 305 may be mounted on and operatively/communicatively coupled to the housing cable 320. In the example of FIG. 3A, the electrode 310A may be a right arm (RA) electrode, the electrode 310B may be a V1 electrode, the electrode 310C may be a V6 electrode, the electrode 310D may be a left arm (LA) electrode, and the electrode 310E may be a left leg (LL) electrode. The electrode 310A and the electrode 310D may each be positioned anywhere on a respective arm of the user from the fingers to the upper chest region by the shoulders (as shown in FIG. 3D). The position of the LL electrode 310E can be anywhere from the toes to the lower left quadrant of the torso near the groin. As shown in FIG. 3A, the computing device 305 and electrodes 310A-310E are implemented in a circular shape, however the computing device 305 and electrodes 310A-310E may be implemented or realized in any appropriate shape and using any appropriate material. Each of the electrodes 310 may be a dry or wet electrode. The dry electrodes may be coupled to or integrated with clamps, suction cups, bands (e.g., wrist bands, leg bands), cuff braces, or any other appropriate coupling mechanism for coupling to a user.

The computing device 305 may comprise hardware to perform the functions described herein. FIG. 3B illustrates a hardware block diagram of computing device 305 which may include hardware such as processing device 306 (e.g., processors, central processing units (CPUs)), memory 307 (e.g., random access memory (RAM), storage devices (e.g., hard-disk drive (HDD)), solid-state drives (SSD), etc.), and other hardware devices (e.g., analog to digital converter (ADC) etc.). A storage device may comprise a persistent storage that is capable of storing data. A persistent storage may be a local storage unit or a remote storage unit. Persistent storage may be a magnetic storage unit, optical storage unit, solid state storage unit, electronic storage units (main memory), or similar storage unit. Persistent storage may also be a monolithic/single device or a distributed set of devices. In some embodiments, such as the one illustrated in FIGS. 3A-3D, the processing device 306 may utilize the electrodes 310 to perform an ECG of a user to which the ECG monitoring device 300 is coupled and then perform basic signal processing of the analog signals measured by each of the electrodes 310 during the ECG. The computing device 305 may include an ADC (not shown) having a high enough sampling frequency for accurately converting the analog signals measured by the set of electrodes 310 into digital signals (e.g., a 24 bit ADC operating at 500 Hz or higher) for processing by the processing device 306. In some embodiments, the ADC may be a component of the processing device 306. The processing device 306 may generate ECG data by calculating the potential difference for each of the set of leads being measured (e.g., leads I, II, V1, and V6). In some embodiments, the processing device 306 may comprise a dedicated ECG waveform processing and analysis chip that provides built-in leads off detection. The processing device 306 may also determine a V6-V1 interpeak interval as discussed in further detail herein.

The computing device 305 may further comprise a transceiver 308, which in some embodiments may implement any appropriate protocol for transmitting the ECG data and/or the V6-V1 interpeak interval to a computing device 405 (shown in FIG. 3D, and which may represent e.g., a cloud service) either directly or via a network. For example, the transceiver 308 may include (or be coupled to) a network interface device (not shown) configured to connect with a network 420 (illustrated in FIG. 3D) to transmit the signal processed data to a computing device 405 either directly or via a mobile computing device (not shown). The network 420 may be a cellular data network (e.g., using GSM, GSM plus EDGE, CDMA, quadband, or other cellular protocols) or a WiFi (e.g., an 802.11 protocol) network. In some embodiments, the transceiver 308 may transmit the ECG data to the mobile computing device (not shown) which may in turn transmit the ECG data and/or the V6-V1 interpeak interval to the computing device 405. For example, the transceiver 308 may comprise a Bluetooth™ chip for transmitting ECG data via Bluetooth to the mobile computing device (e.g., a laptop or smart phone of the user).

In some embodiments, the housing cable 320 may comprise a set of signal cables (shown in FIG. 3D), each of which may couple a respective electrode 310 to the computing device 305. Each of the signal cables may be any appropriate cable which can facilitate the transfer of data between the electrodes 310 and the computing device 305 using any appropriate data transfer protocol such as e.g., universal serial bus (USB). For example, each of the set of signal cables may comprise a USB cable and each of the electrodes 310 may comprise a USB socket (not shown), while the computing device 305 may comprise a USB socket (not shown) to receive data from each of the set of signal cables (where the housing cable 320 provides a common USB plug/output for each of the set of signal cables). It should be noted that in some embodiments, the housing cable 320 may not be removable and may be permanently integrated to the electrodes 310 and the computing device 305. In other embodiments, the housing cable 320 may be removably coupled to the electrodes 310 and the computing device 305.

The positioning of the electrodes 310 allows the user to connect the ECG monitoring device 300 to themselves such that each electrode 310 contacts the appropriate location (e.g., as indicated in and discussed in further detail with respect to FIG. 3C). Once connected to the user, the positioning of the electrodes 310 along the housing cable 320 may allow the user to lay in a relaxed supine position while the ECG monitoring device 300 performs an ECG. This is important because muscular activity (e.g., any kind of motion or use of any muscle) may generate an action potential that is larger than a potential produced by the heart. Thus, if the user is moving around (e.g., exerting force on certain electrodes to maintain contact with parts of their body), the muscular activity of such movement may generate potentials that will drown out the potentials from the heart that is are subject of an ECG. Allowing the user to lay in a relaxed position eliminates any stray potentials (e.g., muscle noise (EMG) or motion artifacts) that could interfere with the signals measured by each electrode 310. Removing the EMG and motion artifacts allows for a cleaner signal and greater diagnostic quality.

FIG. 3C illustrates the ECG monitoring device 300 in operation. As can be seen, each of the electrodes 310 and the computing device 305 may be coupled to the user via any appropriate coupling mechanism. To take an ECG, the user may connect the ECG monitoring device 300 to themselves using the coupling mechanisms as shown to apply each electrode 310 and the computing device 305 to the appropriate location as indicated in FIG. 3C. The ECG monitoring device 300 may connect to the user in such a way as to allow the user to lie down in a relaxed position while an ECG is being taken, which is important for the reasons discussed hereinabove.

The user may position the electrodes 310 such that the electrodes 310 are in contact with the RA, LA, LL, V1, and V6 positions as shown in FIG. 3C. In some examples, a computing device 405 (shown in FIG. 3D) alerts the user to the appropriate location to place the electrodes 310 on the body of the user. In another example, the chest and arm locations are marked and shown by a healthcare professional who prescribes the ECG monitoring device 300 to the patient. In some embodiments, one or more of the electrodes 310 of the ECG monitoring device 300 may comprise dry electrodes, which are easier for at home use because they stay in place more easily and have a longer lifetime use. Dry electrodes tend to provide lower signal quality due to a lower level of conductive contact. In other embodiments, one or more of the electrodes 310 may be wet electrodes, e.g., when the ECG monitoring device 300 is being operated by a healthcare professional or the user has been trained to use wet electrodes. Wet electrodes may provide better conductive contact and produce a higher quality signal. The length of the housing cable 320 may accommodate users of various sizes. The housing cable 320 may have a length that corresponds to (e.g., fits with no slack between electrodes 310) a user with a particular height (e.g., six foot 2 inches), and users that are smaller in height than the height the housing cable 320 corresponds to may simply have additional slack between electrodes once the ECG monitoring device 300 has been coupled to them.

In some embodiments as illustrated in FIG. 3E, the section of the housing cable 320 between electrode 310B and 310C may be sized so that instead of going across the user's chest, the housing cable 320 may travel from the location of the electrode 310B towards the user's groin, and then travel from the user's groin to the location of the electrode 310C. In this way, the housing cable 320 may avoid areas of the user's chest where an x-ray may be taken during e.g., implantation of cardiac pacing leads into the heart of a user.

In some embodiments, the housing cable 320 may have elastic properties and may have a length corresponding to a user with a smaller particular height (e.g., 4 foot 7 inches). The signal cables and the cable 355 may each be implemented using a flexible printed circuit (not shown) with an over mold (not shown) comprising e.g., a thermoplastic polyurethane material or any other appropriate material to provide a conductive channel through which analog signals generated by an electrode 310 may be routed to the computing device 305. In some embodiments, all of the electrodes 310 may be implemented using a single flexible printed circuit that runs the length of the housing cable 320. In these embodiments, the closer the individual is to the height the housing cable 320 corresponds to, the less tension will be applied the housing cable 320.

FIG. 3D illustrates a system block diagram of the ECG monitoring device 300 as shown in FIG. 3A without the housing cable 320. Each of the electrodes 310 may be coupled directly and individually to the computing device 305 via a respective signal cable from the set of signal cables the housing cable 320 is comprised of. When performing an ECG, each electrode 310 may measure electrical activity of the heart from their particular location (e.g., RA, LA, LL, V1, and V6) and transmit the analog signals corresponding to the measured electrical activity to the computing device 305. As discussed herein, the processing device 306 within the computing device 305 may perform basic signal processing of the analog signals received from the electrodes 310 and generate ECG data (by calculating the potential difference for each lead being measured) including a waveform for each lead being measured. The leads being measured may include leads I, II, V1, and V6. The processing device 306 may then transmit the ECG data via the transceiver 308 to a computing device 405 (which may represent e.g., a cloud service).

The computing device 405 may automatically determine the V6-V1 interpeak interval based on the obtained V1 and V6 leads. As shown in FIG. 3D, the computing device 405 may include a V6-V1 interpeak interval analysis module 407 which may be executed by the computing device 405 to help differentiate between different types of ventricular conduction system capture (e.g., between left bundle branch (LBB) capture and left ventricular septal myocardium (LVS) capture), evaluate a level of ventricular conduction system capture, and/or identify transitions between capture types. For example, during LBB area pacing, the various possible combinations of direct capture/non-capture of the septal myocardium and the LBB result in distinct patterns of right and left ventricular activation. This may translate into different combinations of R-wave peak time in the V1 and V6 leads. Thus, the computing device 405 may use the V6-V1 interpeak interval to differentiate between various types of capture e.g., non-selective (ns-)LBB, selective (s-)LBB, and left ventricular septal (LVS).

The computing device 405 may determine the V6-V1 interpeak interval based on a measured interval between the R-wave peak in lead V6 and the R-wave peak in lead V1. ore specifically, the computing device 405 may measure the R-wave peak time (RWPT) in V6 and use the R-wave peak in V1 as an intra-QRS reference time point rather than the pacing stimulus. This circumvents the pacing latency that is present after the pacing stimulus as well as the intraventricular conduction delay related to V6 RWPT prolongation.

The computing device 405 may first measure the V1 RWPT, from the pacing stimulus to the peak of the dominant R wave in lead V1. The computing device 405 may then measure the V6 RWPT, from the pacing stimulus to the peak of R wave in lead V6. Finally, the computing device 405 measures the V6-V1 interpeak interval, from the R-wave peak in lead V6 to the R-wave peak in lead V1. Thus, the V6-V1 interpeak interval is an accurate, easy to measure and reproducible criterion that differentiates non-selective left bundle branch (ns-LBB) capture from left ventricular septal (LVS) myocardial capture.

It should be noted that in some embodiments, the processing device 306 may determine the V6-V1 interpeak interval as discussed above and then transmit the ECG data and the V6-V1 interpeak interval via the transceiver 308, to the computing device 405 for display and further analysis (as discussed in further detail herein).

As discussed hereinabove, a prerequisite for successful cardiac pacing is capture of the ventricular conduction system tissue. However, evaluating the level of capture of the ventricular conduction system tissue can be challenging due to non-selective (ns) capture, which refers to the simultaneous activation of the conduction system and the adjacent myocardium. The use of the V6-V1 interpeak interval, which can be read from an ECG and is a helpful criterion for differentiating between different types of ventricular conduction system capture (e.g., between left bundle branch (LBB) capture and left ventricular septal myocardium (LVS) capture), evaluating a level of ventricular conduction system capture, and/or identifying transitions between capture types.

In one example scenario, during implantation of cardiac pacing leads into the heart of a user (e.g., the His bundle or left bundle branch area), the user may also have the device 300 coupled to them. The device 300 may perform an ECG and transmit the ECG data to the computing device 405 (which, in this example, may represent a computing device of the physician performing the implantation of the cardiac pacing leads). The computing device 405 may calculate the V6-V1 interpeak interval as described herein and display the V6-V1 interpeak interval and related information (as described in further detail herein with respect to FIG. 3F) to the physician at various points during implantation of the cardiac pacing leads. The value of the V6-V1 peak interval tells the physician what area they are activating so that they can ensure that the cardiac pacing leads are implanted such that optimal capture of the ventricular conduction system tissue is achieved (i.e., the cardiac pacing is optimized). The physician may repeat this process several times during implantation of the cardiac pacing leads into the user's heart in order to find the optimal placement for the cardiac pacing leads.

The computing device 405 may also include a conduction system pacing application (CSPA) 421, which may provide a graphical user interface (GUI) via which the V6-V1 interpeak interval and related data calculated during execution of the V6-V1 interpeak interval analysis module 407 may be displayed. An example GUI is shown in FIG. 3F, which illustrates the V1, V6 and aVL leads and the V6-V1 interpeak interval (46 ms) among other information. The CSPA 421 may allow for continuous recording and display of the I, II, V1, and V6 leads, as well as storage of the recorded data in a memory of the computing device 405 in any appropriate format (e.g., JSON). The CSPA 421 may provide running median beats of V1 and V6 over any appropriate interval (e.g., running five-second median beats of V1 and V6) and may also provide manual cursors that provide the time in milliseconds between them that can be applied to median beats or the recorded individual beats for the measurement of the following parameters: left ventricular activation time (LVAT), stimulation to peak of the dominant R wave in lead V1 duration (V1: R), V6-V1 interpeak interval, stimulation to peak of the dominant R wave in lead V6 duration (LVCT-LV Conduction Time), and QRS duration (V1 and V6). As shown in FIG. 3F, each of the LVAT, V1: R, V6-V1, and QRS values may be an interactable GUI element (e.g., a button) that the physician may tap/press on to select the cursor to adjust for that particular measurement. Alternatively, the physician may tap “Calipers” for general calipers. The CSPA 421 may also provide the ability to print the GUI display to any document format (e.g., Word or PDF) in order to save the measured values in a document format. The CSPA 421 may also provide the ability to adjust the gain and sweep speed by tapping on the scale settings at the bottom of the GUI (e.g., tap “100 mm/s” to cycle between 25, 50, 100 mm/s sweep speeds and/or tap “40 mm/v” to cycle between 5, 10, 20, and 40 mm/mv).

FIG. 3G illustrates the three types of capture during left bundle branch area pacing (LBBP) observed in a patient during pacemaker implantation, where leads V1 and V6 are recorded simultaneously. During the transition from non-selective LBB-P to selective LBB-P, there is an increase in V1 R-wave peak time (V1RWPT) but not in V6RWPT, resulting in a wider V6-V1 interpeak distance. In contrast, during the transition from non-selective LBB-P to left ventricular septal (LVS) myocardial capture there is an increase in V6RWPT but not in V1RWPT, resulting in a shorter V6-V1 interpeak distance.

The computing device 405 may also include a lead synthesis software module 410 and an ECG waveform interpretation software module 415. The computing device 405 may execute the lead synthesis software 410 to synthesize (based on the received ECG data) ECG waveforms corresponding to leads that were not measured by the electrodes 310 of the ECG monitoring device 300 as discussed in further detail herein. The computing device 405 may execute the ECG waveform interpretation software 415 to generate diagnostic interpretations based on the measured and synthesized ECG waveforms, as discussed in further detail herein. After the computing device 405 has executed the lead synthesis software 410 and the ECG waveform interpretation software 415, it may transmit the results to a computing device (e.g., computing device of a physician or healthcare provider, or a mobile device/laptop of the user).

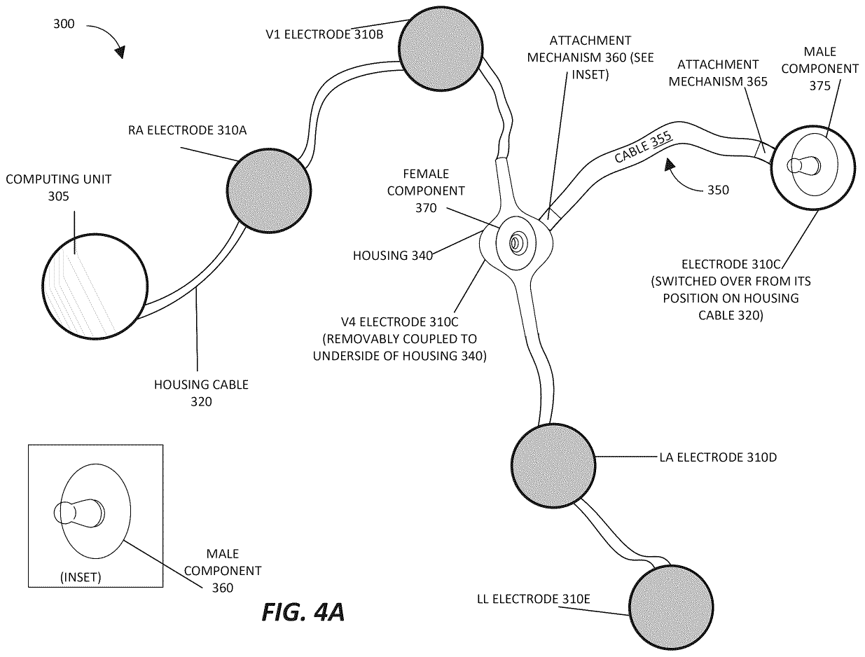

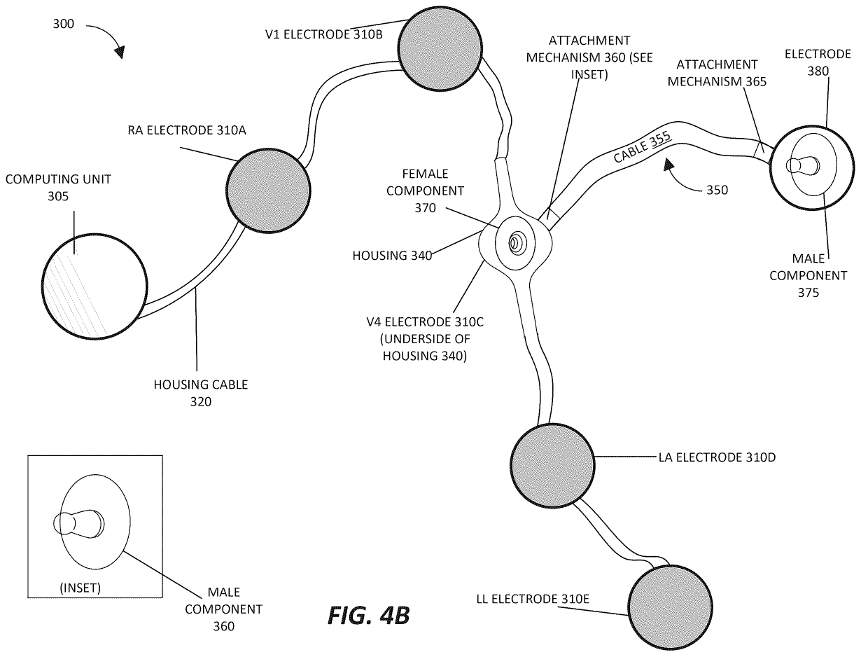

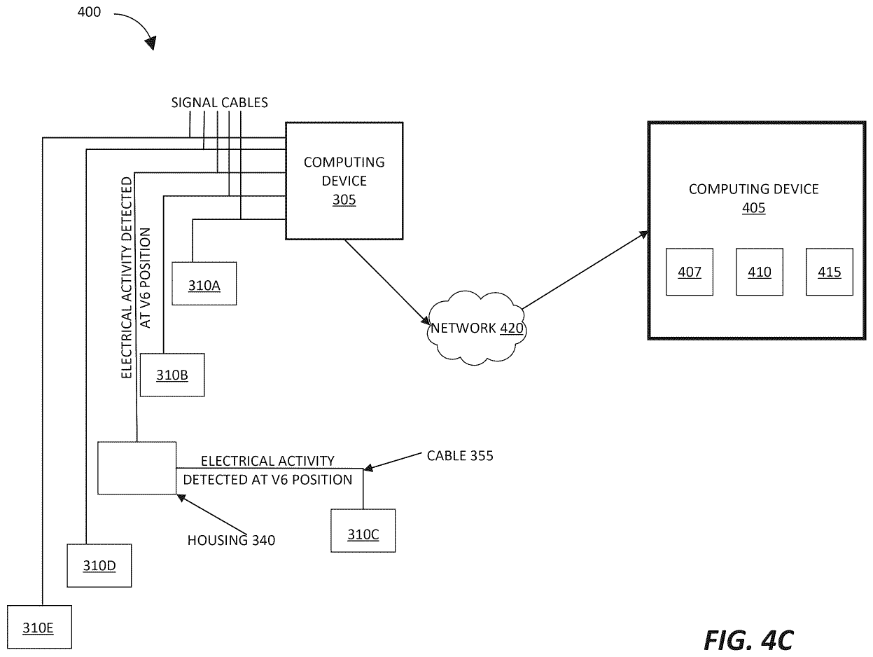

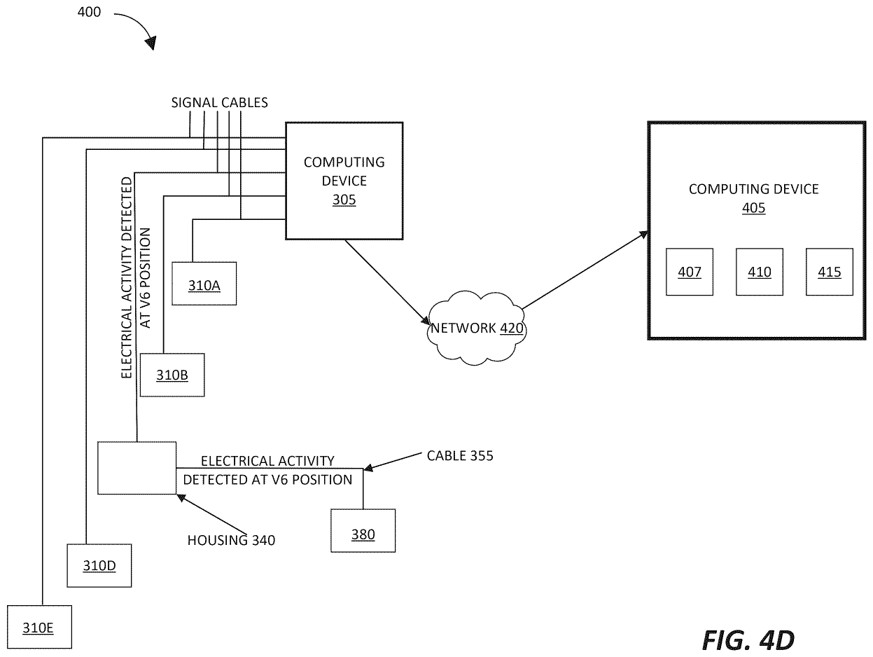

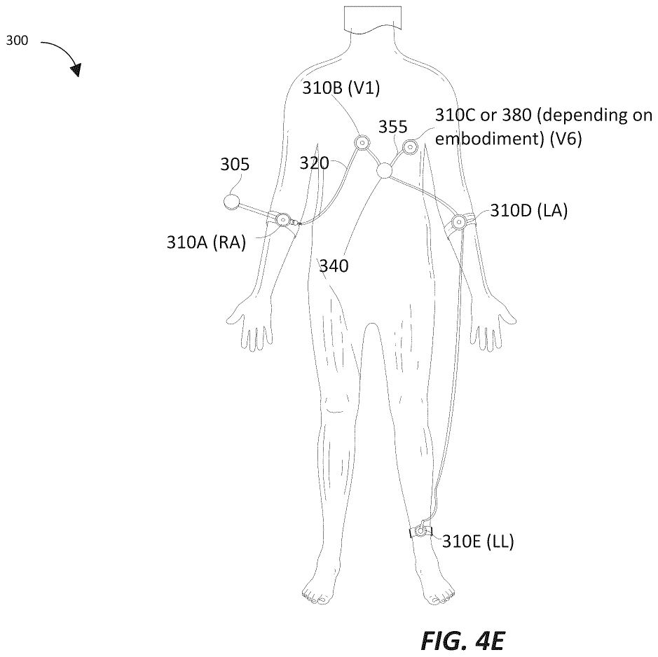

Referring now to FIG. 4A, in some embodiments where the electrode 310C is a V4 electrode, the electrode 310C may be implemented within a housing 340 that is communicatively coupled to both the electrode 310C and the housing cable 320 (and thus communicatively couples the electrode 310C to the housing cable 320). Via the housing 340, the housing cable 320 may be connected to a lead extender 350 as shown in FIG. 4A. The lead extender 350 may comprise a cable 355 having a first attachment mechanism 360 and a second attachment mechanism 365. The first attachment mechanism 360 may couple the cable 355 to the housing 340, and thus to the housing cable 320 (as the housing 340 and housing cable 320 are already communicatively coupled). In some embodiments, the first attachment mechanism 360 may comprise a male component of a snap rivet (shown inset in FIG. 4A) and may also be referred to herein as male component 360, while the housing 340 may comprise a female component 370 of the snap rivet. Alternatively, the housing 340 may comprise the male component while the first attachment mechanism 360 may comprise the female component. Under pressure, the male component 360 is pushed through a hole in the female component 370 or pushed into a depression in the female component 370 to securely couple the male and female components together. The male component 360 and the female component 370 may both be conductive, allowing for the cable 355 to be communicatively coupled to the housing cable 320 via the housing 340 when the male component 360 and the female component 370 are coupled as described herein. The second attachment mechanism 365 may comprise a female or male component of a snap rivet and for the purposes of the example of FIG. 4A may be referred to herein as female component 365. The cable 355 may be sized so that an electrode that is connected to the female component 365 may contact the V6 position (mid-axillary line of the 5^{th} intercostal space) of the user.

In some embodiments, the electrode 310C may have a male or female component of a snap rivet (in the example of FIG. 4A, a male component), enabling it to be removably attached to the housing 340 in a manner similar to the manner in which the cable 355 is attached to the housing 340 as discussed herein. When a V6-V1 interpeak interval measurement is desired (as discussed in further detail herein), the electrode 310C may be removed from the housing 340 and its male component is pushed through a hole in the female component 365 or pushed into a depression in the female component 365 so that it is removably attached to the cable 355 and can contact the V6 position. Because the male component of the electrode 310C and the female component 365 are both conductive, the electrode 310C may be communicatively coupled to the cable 355. In this way, no additional electrodes are necessary in order to obtain the V6-V1 interpeak interval.

Referring now to FIG. 4B, in other embodiments, the female component 365 may be coupled to a male component 375 of a snap rivet that is mounted on an electrode 380. The electrode 380 may be an electrode that is additional to the set of electrodes 310. The male component 375 and the female component 365 may both be conductive, allowing for the cable 355 to be communicatively coupled to the electrode 380 when the male component 375 and the female component 365 are coupled. As discussed hereinabove, the cable 355 may be sized to allow the electrode 380 to contact the V6 position (mid-axillary line of the 5^{th} intercostal space) of a user. In this way, no switching of electrodes 310 is required in order to obtain the V6-V1 interpeak interval.

As discussed herein, the computing device 305 may comprise hardware to perform the functions described herein. In some embodiments such as the one illustrated in FIGS. 4A, 4C and 4E, the processing device 306 may utilize the electrodes 310 to perform an ECG of a user to which the ECG monitoring device 300 is coupled and then perform basic signal processing of the analog signals measured by each of the electrodes 310. The ECG may be performed with the electrode 310C switched to the V6 position, as discussed hereinabove. In other embodiments, such as the one illustrated in FIGS. 4B, 4D and 4F, the processing device 306 may utilize the electrodes 310 and the electrode 380 to perform an ECG of a user to which the ECG monitoring device 300 is coupled and then perform basic signal processing of the analog signals measured by each of the electrodes 310 and 380. The ECG may be performed with the electrode 380 in the V6 position as discussed hereinabove and the electrode 310C disconnected from the housing 340 (and thus the housing cable 320). In some embodiments, the electrode 310C may remain connected to the housing 340. The positioning of the electrodes 310 and the electrode 380 allows the user to connect the ECG monitoring device 300 to themselves such that each electrode 310 and the electrode 380 contacts the appropriate location (e.g., as indicated in and discussed in further detail with respect to FIG. 4E).

The processing device 306 may generate ECG data by calculating the potential difference for each of the set of leads being measured (e.g., leads I, II, V1, and V6). In some embodiments, the processing device 306 may comprise a dedicated ECG waveform processing and analysis chip that provides built-in leads off detection. The processing device 306 may then transmit the ECG data via the transceiver 308 to a computing device 405 (which may represent e.g., a cloud service). The computing device 405 may automatically determine the V6-V1 interpeak interval based on the obtained V1 and V6 leads as discussed in further detail hereinabove.

The positioning of the electrodes 310 allows the user to connect the ECG monitoring device 300 to themselves such that each electrode 310 (and electrode 380 in some embodiments) contacts the appropriate location (e.g., as indicated in and discussed in further detail with respect to FIGS. 4E and 4F). Once connected to the user, the positioning of the electrodes 310 (and electrode 380 in some embodiments) along the housing cable 320 and the cable 355 may allow the user to lay in a relaxed supine position while the ECG monitoring device 300 performs an ECG. This is important because muscular activity (e.g., any kind of motion or use of any muscle) may generate an action potential that is larger than a potential produced by the heart. Thus, if the user is moving around (e.g., exerting force on certain electrodes to maintain contact with parts of their body), the muscular activity of such movement may generate potentials that will drown out the potentials from the heart that are the subject of an ECG. Allowing the user to lay in a relaxed position eliminates any stray potentials (e.g., muscle noise (EMG) or motion artifacts) that could interfere with the signals measured by each electrode 310 (and electrode 380 in some embodiments).

FIG. 4C illustrates a system block diagram of the ECG monitoring device 300 as shown in FIG. 4A without the housing cable 320. Each of the electrodes 310 may be coupled directly and individually to the computing device 305 via a respective signal cable from the set of signal cables the housing cable 320 is comprised of except for electrode 310C. Electrode 310C has been switched to the V6 position and is attached to the cable 355 via the attachment mechanism 365. Via the cable 355, the electrode 310C is coupled to the housing 340, which in turn connects the electrode 310C to a respective signal cable from the set of signal cables the housing cable 320 is comprised of as discussed in further detail herein. When performing an ECG, each electrode 310 may measure electrical activity of the heart from their particular location (e.g., RA, LA, LL, V1, and V6) and transmit the analog signals corresponding to the measured electrical activity to the computing device 305. The housing 340 may provide the analog signals corresponding to the measured electrical activity at the V6 location to the computing device 305 via the respective signal cable of the electrode 310C. As discussed herein, the processing device 306 within the computing device 305 may perform basic signal processing of the analog signals received from the electrodes 310 and generate ECG data (by calculating the potential difference for each lead being measured) including a waveform for each lead being measured. The leads being measured may include leads I, II, V1, and V6. Because the ECG monitoring device 300 includes the lead extender 350, the processing device 306 may also receive analog signals corresponding to the electrical activity of the user at the V6 position, allowing it to obtain the lead V6.

The processing device 306 may then transmit the ECG data via the transceiver 308 to a computing device 405 which may automatically determine the V6-V1 interpeak interval based on the obtained V1 and V6 leads as discussed hereinabove.

FIG. 4D illustrates a system block diagram of the ECG monitoring device 300 as shown in FIG. 4B without the housing cable 320. Each of the electrodes 310 may be coupled individually to the computing device 305 via a respective signal cable from the set of signal cables the housing cable 320 is comprised of except for electrode 310C which is disconnected. Electrode 380 is attached to the cable 355 via the attachment mechanism 365. Via the cable 355, the electrode 380 is coupled to the housing 340, which in turn connects the electrode 380 to a respective signal cable from the set of signal cables the housing cable 320 is comprised of as discussed in further detail herein. When performing an ECG, each electrode 310 and the electrode 380 may measure electrical activity of the heart from their particular location (e.g., RA, LA, LL, V1, and V6) and transmit the analog signals corresponding to the measured electrical activity to the computing device 305. The electrode 380 may measure the electrical activity of the heart from the V6 location and provide analog signals corresponding to the measured electrical activity at the V6 location to the housing 340 via the cable 355. The housing 340 may provide the analog signals corresponding to the measured electrical activity at the V6 location to the computing device 305 via the respective signal cable of the electrode 310C (which is disconnected). As discussed herein, the processing device 306 within the computing device 305 may perform basic signal processing of the analog signals received from the electrodes 310 and 380, generate ECG data (by calculating the potential difference for each lead being measured) including a waveform for each lead being measured. The leads being measured may include leads I, II, V1, and V6.

The processing device 306 may then transmit the ECG data via the transceiver 308 to a computing device 405 which may automatically determine the V6-V1 interpeak interval based on the obtained V1 and V6 leads as discussed hereinabove. It should be noted that in some embodiments, the processing device 306 may determine the V6-V1 interpeak interval as discussed above and then transmit the ECG data and the V6-V1 interpeak interval via the transceiver 308, to the computing device 405 for display and further analysis.

Referring to both FIGS. 4C and 4D, the computing device 405 may include a V6-V1 interpeak interval analysis module 407, a lead synthesis software module 410 and an ECG waveform interpretation software module 415, each of which may be executed by the computing device 405 to perform their respective functions as described hereinabove.

FIG. 4E illustrates the ECG monitoring device 300 (of FIGS. 4A-4D) in operation. As can be seen, each of the electrodes 310 (and the electrode 380 depending on the embodiment) and the computing device 305 may be coupled to the user via any appropriate coupling mechanism. To take an ECG, the user may connect the ECG monitoring device 300 to themselves using the coupling mechanisms as shown to apply each electrode 310 (and the electrode 380 depending on the embodiment) and the computing device 305 to the appropriate location as indicated in FIG. 4E. The ECG monitoring device 300 may connect to the user in such a way as to allow the user to lie down in a relaxed position while an ECG is being taken, which is important for the reasons discussed hereinabove.

The user may position the electrodes 310 (and 380 depending on the embodiment) such that electrodes are in contact with the RA, LA, LL, V1, and V6 positions as shown in FIG. 4E. In some examples, a mobile computing device 405 alerts the user to the appropriate location to place the electrodes 310 (and 380 depending on the embodiment) on the body of the user. In another example, the chest and arm locations are marked and shown by a healthcare professional who prescribes the ECG monitoring device 300 to the patient. In some embodiments, one or more of the electrodes 310 (and 380 depending on the embodiment) of the ECG monitoring device 300 may comprise dry electrodes, which are easier for at home use because they stay in place more easily and have a longer lifetime use. Dry electrodes tend to give lower signal quality due to a lower level of conductive contact. In other embodiments, one or more of the electrodes 310 (and 380 depending on the embodiment) may be wet electrodes, e.g., when the ECG monitoring device 300 is being operated by a healthcare professional or the user has been trained to use wet electrodes. The wet electrodes may provide better conductive contact and produce a higher quality signal. The length of the housing cable 320 may accommodate users of various sizes. The housing cable 320 may have a length that corresponds to (e.g., fits with no slack between electrodes 310) a user with a particular height (e.g., six foot 2 inches), users that are smaller in height than the height the housing cable 320 corresponds to may simply have additional slack between electrodes once the ECG monitoring device 300 has been coupled to them.

In some embodiments, the housing cable 320 may have elastic properties and may have a length corresponding to a user with a smaller particular height (e.g., 4 foot 7 inches). The signal cables and the cable 355 may each be implemented using a flexible printed circuit (not shown) with an over mold (not shown) comprising e.g., a thermoplastic polyurethane material or any other appropriate material to provide a conductive channel through which analog signals generated by an electrode 310 or 380 may be routed to the computing device 305. In some embodiments, all of the electrodes 310 may be implemented using a single flexible printed circuit that runs the length of the housing cable 320. In these embodiments, the closer the individual is to the height the housing cable 320 corresponds to, the less tension will be applied the housing cable 320.

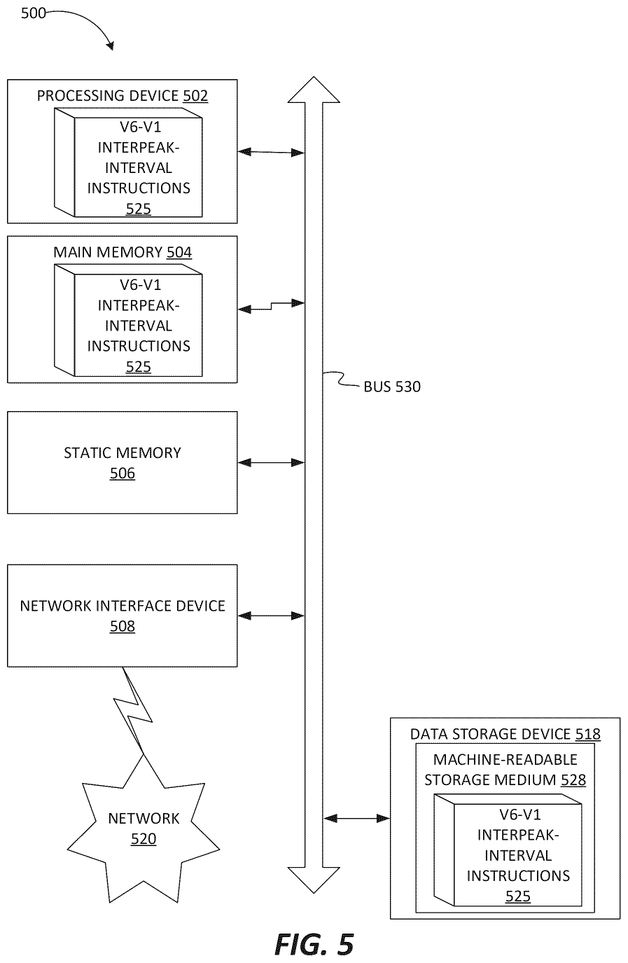

FIG. 5 illustrates a diagrammatic representation of a machine in the example form of a computer system 500 within which a set of instructions, for causing the machine to perform any one or more of the methodologies discussed herein for determining a V6-V1 interpeak interval, in accordance with some embodiments of the present disclosure.

In alternative embodiments, the machine may be connected (e.g., networked) to other machines in a local area network (LAN), an intranet, an extranet, or the Internet. The machine may operate in the capacity of a server or a client machine in a client-server network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a server, a network router, a switch or bridge, a hub, an access point, a network access control device, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. In one embodiment, computer system 500 may be representative of a computing device, such as computing device 305.

The exemplary computer system 500 includes a processing device 502, a main memory 504 (e.g., read-only memory (ROM), flash memory, dynamic random access memory (DRAM), a static memory 506 (e.g., flash memory, static random access memory (SRAM), etc.), and a data storage device 518, which communicate with each other via a bus 530. Any of the signals provided over various buses described herein may be time multiplexed with other signals and provided over one or more common buses. Additionally, the interconnection between circuit components or blocks may be shown as buses or as single signal lines. Each of the buses may alternatively be one or more single signal lines and each of the single signal lines may alternatively be buses. Computing device 500 may further include a network interface device 508 which may communicate with a network 520.

Processing device 502 represents one or more general-purpose processing devices such as a microprocessor, central processing unit, or the like. More particularly, the processing device may be complex instruction set computing (CISC) microprocessor, reduced instruction set computer (RISC) microprocessor, very long instruction word (VLIW) microprocessor, or processor implementing other instruction sets, or processors implementing a combination of instruction sets. Processing device 502 may also be one or more special-purpose processing devices such as an application specific integrated circuit (ASIC), a field programmable gate array (FPGA), a digital signal processor (DSP), network processor, or the like. The processing device 502 is configured to execute V6-V1 interpeak interval instructions 525, for performing the operations and steps discussed herein.

The data storage device 518 may include a machine-readable storage medium 528, on which is stored one or more sets of V6-V1 interpeak interval instructions 525 (e.g., software) embodying any one or more of the methodologies of functions described herein. The V6-V1 interpeak interval instructions 525 may also reside, completely or at least partially, within the main memory 504 or within the processing device 502 during execution thereof by the computer system 500; the main memory 504 and the processing device 502 also constituting machine-readable storage media. The V6-V1 interpeak interval instructions 525 may further be transmitted or received over a network 520 via the network interface device 508.

While the machine-readable storage medium 528 is shown in an exemplary embodiment to be a single medium, the term “machine-readable storage medium” should be taken to include a single medium or multiple media (e.g., a centralized or distributed database, or associated caches and servers) that store the one or more sets of instructions. A machine-readable medium includes any mechanism for storing information in a form (e.g., software, processing application) readable by a machine (e.g., a computer). The machine-readable medium may include, but is not limited to, magnetic storage medium (e.g., floppy diskette); optical storage medium (e.g., CD-ROM); magneto-optical storage medium; read-only memory (ROM); random-access memory (RAM); erasable programmable memory (e.g., EPROM and EEPROM); flash memory; or another type of medium suitable for storing electronic instructions.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

While preferred embodiments of the present disclosure have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An apparatus comprising:
a housing cable;
a set of electrodes positioned along the housing cable so that each of the set of electrodes contacts a particular location of a user when the set of electrodes are connected to the user; and
a computing device positioned along the housing cable and operatively coupled to each of the set of electrodes via the housing cable, the computing device comprising:
a memory; and
a processing device operatively coupled to the memory, the processing device to:
receive analog signals corresponding to electrical activity measured by each of the set of electrodes;
generate, based on the analog signals, a set of leads including a V1 lead and a V6 lead; and
determine a V6-V1 interpeak interval based on the V1 lead and the V6 lead.

2. The apparatus of claim 1, wherein the housing cable comprises a set of signal cables positioned therein, and wherein the computing device is operatively coupled to each of the set of electrodes via a respective signal cable of the set of signal cables.

3. The apparatus of claim 1, wherein the computing device further comprises:
a transceiver to transmit the V6-V1 interpeak interval to a mobile computing device.

4. The apparatus of claim 1, wherein the particular location contacted by each of the set of electrodes corresponds to a right arm of the user, a left arm of the user, a left leg of the user, a V1 chest position of the user, and a V6 chest position of the user respectively.

5. The apparatus of claim 1, wherein each of the set of signal cables comprises a flexible printed circuit.

6. The apparatus of claim 1, wherein the housing cable comprises an elastic material.

7. The apparatus of claim 1, wherein each of the set of electrodes comprises a coupling mechanism to connect the electrode to a corresponding respective location of the user.

8. The apparatus of claim 7, wherein each of the coupling mechanisms comprises one of: a clamp, a suction cup, a band, and a cuff brace.

9. The apparatus of claim 1, wherein each of the set of electrodes comprises an adhesive material to connect the electrode to the corresponding particular location of the user.

10. The apparatus of claim 1, wherein each of the set of electrodes contacts the corresponding particular location of the user without any muscular activity of the user when the set of electrodes are connected to the user.

11. A system comprising:
a mobile computing device; and
an apparatus comprising:
a housing cable;
a set of electrodes positioned along the housing cable so that each of the set of electrodes contacts a particular location of a user when the set of electrodes are connected to the user; and
a computing device positioned along the housing cable and operatively coupled to each of the set of electrodes via the housing cable, the computing device comprising:
a memory; and
a processing device operatively coupled to the memory, the processing device to:
receive analog signals corresponding to electrical activity measured by each of the set of electrodes; and
transmit the analog signals to the mobile computing device, wherein the mobile computing device is to:
generate, based on the analog signals, a set of leads including a V1 lead and a V6 lead;
determine a V6-V1 interpeak interval based on the V1 lead and the V6 lead; and
display the V6-V1 interpeak interval.

12. The system of claim 11, wherein the housing cable comprises a set of signal cables positioned therein, and wherein the computing device is operatively coupled to each of the set of electrodes via a respective signal cable of the set of signal cables.

13. The system of claim 11, wherein the computing device further comprises:
a transceiver via which the processing device transmits the analog signals to the mobile computing device.

14. The system of claim 11, wherein the particular location contacted by each of the set of electrodes corresponds to a right arm of the user, a left arm of the user, a left leg of the user, a V1 chest position of the user, and a V6 chest position of the user respectively.

15. The system of claim 11, wherein each of the set of signal cables comprises a flexible printed circuit.

16. The system of claim 11, wherein to display the V6-V1 interpeak interval, the mobile computing device is to:
display a graphical user interface (GUI) that provides an indication of:
running median beats for each of the set of leads over an interval;
the V6-V1 interpeak interval; and
a QRS duration.

17. The system of claim 16, wherein each of the indications comprises an interactable GUI element that provides cursors to adjust information corresponding to the indication.

18. The system of claim 17, wherein the GUI further provides, for each of a gain and a sweep speed, an interactable element to adjust the gain and the sweep speed respectively.

19. The system of claim 11, wherein each of the set of electrodes comprises an adhesive material to connect the electrode to the corresponding particular location of the user.

20. The system of claim 11, wherein each of the set of electrodes contacts the corresponding particular location of the user without any muscular activity of the user when the set of electrodes are connected to the user.
